(19) United States
(12) Patent Application Publication
(10) Pub. No.: US 2002/0022743 A1
(43) Pub. Date: Feb. 21, 2002

(54) Method for the purification of aryl sulfonic acids and salts

(76) Inventors: Sergei Pouhov, Fremont, CA (US); Gene Jamieson, Boulder Creek, CA (US); John Vajda, Sunnyvale, CA (US); Henry Kruk, San Jose, CA (US); John McGinley, San Francisco, CA (US)

Correspondence Address: William H. Benz, BURNS, DOANE, SWECKER & MATHIS, L.L.P., P.O. Box 1404, Alexandria, VA, 22313-1404, US

(21) Appl. No.: 09/755,619

(22) Filed: Jan. 5, 2001

Related U.S. Application Data
(60) Provisional application No. 60/175,314, filed on Jan. 10, 2000.

(51) Int. Cl. C 07C 3 9/29

(52) U.S. Cl. 562/96

(57) ABSTRACT

Solid aryl sulfonates are purified by preferentially dissolving impurities into an aqueous liquid phase.

### BACKGROUND OF THE INVENTION

1. Field of the Invention

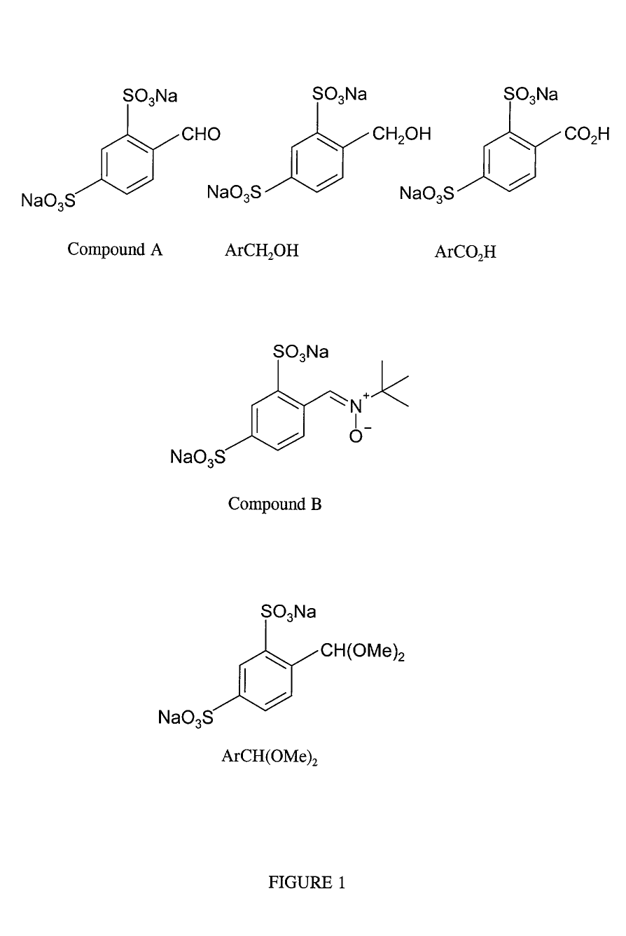

This invention relates to a method for purifying aryl sulfonic acids and salts. Background Materials U.S. Pat. No.5,488,145 discloses the aryl sulfonic acid, α-(2,4-disulfophenyl)-N-tert-butylnitrone, its pharmaceutically acceptable salts, and related pharmaceutical compositions. U.S. Pat. No.5,475,032 discloses the use of such compositions in the treatment of stroke and of progressive central nervous system function loss conditions. U.S. Pat. No. 5,508,305 discloses the use of such compositions for ameliorating the side effects caused by oxidative damage resulting from antineoplastic disease treatment. Similar disclosures are also made in WO 95/17876. U.S. Pat. No. 5,780,510 discloses the use of these same compounds in the treatment of concussion. This most preferred material of this class of compounds is identified as “Compound B” in FIG. 1.

Various methods are available for the synthesis of these materials. The most often used method involves the condensation reaction of a hydroxylamine derivative with an appropriate aldehyde or ketone (J. S. Roberts in D. H. R. Barton and W. D. Ollis, Comprehensive Organic Chemistry, Volume 2, pp. 500-504, Pergamon Press, 1979; R. D. Hinton and E. G. Janzen, J. Org. Chem., 1992, 57, pp. 2646-2651). This general methodology is employed in the above-described patents where the preparation of a-(2,4-disulfophenyl)-N-tert-butylnitrone is described as involving the reaction of the aryl sulfonic acid, 4-formyl-1,3-benzenesulfonic acid (“Compound A” in FIG. 1), with N-tert-butylhydroxylamine in refluxing methanol for approximately 18 hours. One can add an acidic catalyst, if desired. Alternatively one can use an acid addition salt such as the acetic acid, acid addition salt.

In other examples of this synthetic scheme α-(2-sulfophenyl)-N-tert-butylnitrone has been prepared by reaction of 2-formylbenzenesulfonic acid sodium salt with N-tert-butylhydroxylamine in refluxing ethanol for 2 days (E. G. Janzen and R. V. Shetty, Tetrahedron Letters, 1979, pp. 3229-3232) and a modification of this type of methodology for the manufacture α-phenyl-N-methylnitrone has been described in French Patent 1.437,188 to E.I. DuPont de Nemours and Co.

In all of these cases aryl sulfonic acids and salts are present as starting materials and as final products. In all these cases, these aryl sulfonates are solids that have a high degree of water solubility. These materials are commonly accompanied by contaminants and byproducts which often are themselves salts which share this high degree of water solubility. Particularly in cases where the final products are used as pharmaceuticals, their purity and the purity of the feedstocks used to prepare them is of utmost importance.

There are a limited number of methods available to purify aryl sulfonic acids and salts and to isolate these salts from contaminants and byproduct salts. Two common methods include ion exchange chromatography and recrystallization. These methods are not always successful due to a limited choice of solvents available to solubilize the compounds. Aryl sulfonic acid salts such as 4-formyl-1, 3-benzenedisulfonic acid, disodium salt (2,4-DSAD-Na, Compound A), are often contaminated with related aryl impurities, such as the corresponding benzyl alcohol (ArCH₂OH) and benzoic acid (ArCO₂H), as well as inorganic impurities, such as sodium chloride (NaCl). The purification of mixtures of such salts, especially on large scale, is often difficult due to the similar solubilities of the contaminants and the desired products.

Statement of the Invention

We have now found a method for purifying aryl sulfonates (acids and salts). In this method, a slurry of the aryl sulfonate to be purified is stirred in an aqueous solvent system. The aqueous solvent system can be water alone or can be a mixture of water with a nonaqueous carrier liquid. When small amounts of compound are purified a carrier solvent is preferable in order to improve the ease of handling the material. As well, when purifying small amounts of material without using an additional carrier solvent, the yields are generally lower because more water is required to form a workable slurry and to transfer the material.

The purification relies on a mass effect in which the aryl salt to be purified and impurities will be solubilized to their respective saturation limits. Due to a mass effect, impurities present in small amounts, such as up to 15 molar % of the total solid, will not reach their saturation limits while the aryl salt to be purified is present in a larger amount and will reach its saturation limit. This means a higher percentage of the total impurities will be solubilized than the aryl salt to be purified. Thus, on a relative basis, the aryl salt to be purified is solubilized to a lesser extent than those components present in smaller amounts and purification of the solid phase takes place. The resulting suspension is filtered and the solubilized impurities are removed in the filtrate resulting in enhanced purity for the filtered solid.

Thus, in one aspect, this invention provides a method for purifying a material represented generally by structural formula I

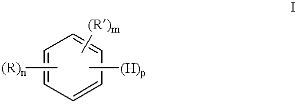

wherein n is an integer from 1 through 3 inclusive, m is an integer from 1 through 3 inclusive and p is an integer 0 through 4 inclusive, with the sum of n +m +p equal to 6, and wherein each R independently represents SO₃H or a salt thereof, and each R is an organic substituting group;

which method comprises obtaining the aryl sulfonate of formula I as an impure particulate solid containing up to 15% molar impurities;

forming a slurry of the impure particulate solid in a liquid phase comprising water, the composition and quantity of liquid phase selected to dissolve not more than ⅔ of the impure particulate solid;

agitating the slurry;

phase separating the slurry into a purified solid phase comprising purified aryl sulfonate and a loaded liquid phase comprising dissolved aryl sulfonate and dissolved impurities; and

recovering the purified solid phase.

In another aspect, the invention provides the purified aryl sulfonic acid materials which are produced by this method.

### DESCRIPTION OF THE PREFERRED EMBODIMENTS

### BRIEF DESCRIPTION OF THE DRAWING

This invention will be further described with reference being made to the drawing in which the chemical structures of certain compounds purified by this method and their impurities are shown.

The Materials being Purified

The materials purified by the present method are aryl sulfonates. These materials are represented generally by structural formula I

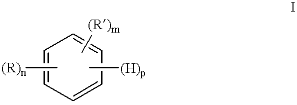

wherein n is an integer from 1 through 3 inclusive, m is an integer from 1 through 3 inclusive and p is an integer from 0 through 4 inclusive, with the sum of n+m+p equal to 6, and wherein each R independently represents SO₃H or a salt thereof, and each R′ is an organic substituting group.

In these materials, n is most commonly 1 or 2, and in our most preferred embodiment is 2; m is 1 or 2 and most preferably is 1.

The R group or groups include SO₃H and salts thereof, represented together as SO₃X, wherein X is hydrogen or one or more pharmaceutically acceptable cations. The cation may be a monovalent material such as sodium, potassium, lithium, ammonium, alkylammonium or diethanolammonium. Alternatively, it may be a polyvalent cation such as calcium, magnesium, aluminum or zinc. It may also be a mixed salt formed with a polyvalent cation such as calcium or magnesium in combination with a pharmaceutically acceptable anion such as halide (for example chloride), phosphate, sulphate, acetate, citrate or tartrate.

The various R's in this formula are usually the same. However, they can be independently selected from the possibilities just enumerated.

It is preferred that there be two R's in formula (I) above, that they be the same and that each represents SO₃ ⁻Na⁺.

The R′ group or groups are organic substituents covalently bonded to the aryl ring. These R′ groups may be hydrocarbyl groups such as alkyl groups, for example, alkyls of 1 to about 10 carbons. More commonly, R′ groups are selected from heteroatom containing organic substituents such as oxyhydrocarbyls such as alcohols, aldehydes, ketones, carboxyls, esters, ethers and the like.

The R′ groups can also be nitrohydrocarbyls such as amines and the like and can also be more complex structures such as nitrones and the like. The identity of R′ should not e considered to be critical so long as R′ permits the compound being purified to be a crystalline solid with significant (e.g., at least about 0.05 g/ml) solubility in water.

Possible families of such compounds include, but are not limited to: substituted polyaromatic compounds with either linear or non-linear fused ring systems such as naphthalene, anthracene and chrysene; substituted polyaromatic compounds without fused ring systems, such as biphenyl; and, substituted aromatic compounds containing heteroatoms, such as pyridine and furan.

In preferred applications, the purification procedure has been used to purify 4-formyl-1,3-benzenedisulfonic acid disodium salt hydrate (2,4-DSAD-Na), in which the levels of an inorganic impurity, NaCl, and structurally related impurities, such as the benzyl alcohol (ArCH₂OH) and the benzoic acid (ArCO₂H) were generally reduced. Similar success was achieved on the related sodium disulfonate nitrone compound, namely, disodium α-(2,4 disulfophenyl)-N-tert butylnitrone. Purification of this compound using the slurry method not only reduced the levels of the NaCl, 2,4-DSAD-Na, ArCH₂OH, and ArCO₂H impurities but also that of a fifth process related impurity, the corresponding dimethyl acetal (ArCH(OMe)₂). These compounds are shown in FIG. 1.

Process Parameters

In the first step of this process, the solid to be purified is slurried in an aqueous solvent system. The amount and composition of this solvent system are controlled so that no more than ⅔, and preferably no more than ½, and especially ⅕ to ⅓ of the desired material being purified is dissolved.

In many cases, water alone may be used as the solvent system in which case the amount of water is controlled to give the desired levels of dissolution. In other cases, one or more additional liquids are added. The function of these additional liquids is to act as a carrier phase and provide a volume of liquid which will provide a workable slurry of solid in liquid as opposed to essentially a solid mass.

The additional carrier liquid can be selected from water-miscible and water-immiscible liquids. The primary limitations on the selection of this liquid are that it be easily removable from the final purified product and that it not react with water or the product or byproducts. In the case of pharmaceutical final products it is also important that trace amounts of the carrier liquid not rouse toxicity and safety concerns.

The carrier liquid can be selected from organic liquids such as liquid hydrocarbons, liquid oxyhydrocarbons and the like. Inorganic liquids such as the liquid fluorocarbons could also be used.

Preferred materials are volatile so that they can be removed by drying processes. Suitable volatility is typically achieved with organic (hydrocarbon and oxyhydrocarbon) liquids having from about one to about five carbon atoms.

Thus, examples of suitable carrier liquids include hydrocarbons such as butane or pentane and oxyhydrocarbons such as lower alcohols of up to four carbons atoms including methanol, ethanol, the propanols and the butanols; ketones of up to four carbon atoms such as acetone, methyl-ethyl ketone and the like; ethers of up to about five carbon atoms, such as diethyl ether; and esters of up to about five carbon atoms such as ethyl acetate and the like. As will be noted, many of these liquids such as the lower alcohols and ketones are water-miscible, while other materials such as the ethers and esters and hydrocarbons are water-immiscible.

In this first step, the solid is slurried in the liquid phase. This is typically done with agitation. The slurrying is continued for a substantial period such as at least about 5 minutes and up to about 48 hours. Most commonly, the slurrying is continued for from about 10 minutes to about 24 hours.

The progress of the purification can be monitored analytically if desired. If desired, an inert gas cap can be applied during slurrying. The slurrying is usually carried out at ambient temperature.

Following the slurrying, the purified solid is phase separated from the contaminant-rich liquid phase. This step can be carried out in a simple settler or filter may be carried out in a centrifuge, a rotary (drum) filter or the like. If desired, a minor amount of wash liquid can be added to the solid after or during the phase separation step.

In the next step, residual liquid is removed from the purified solid. This is typically done by drying the solid. A dry gas stream (air, nitrogen, or the like) can be used as a drying medium. Vacuum and elevated temperatures can be employed. Representative vacuums are vacuums to a pressure of about 0.1 ATM absolute and representative elevated temperatures range from about ambient temperature (20° C.) up to about 110° C. with temperatures of from about 35° C. to about 100° C. being preferred. The drying can be carried out in tray driers or tumbling driers or the like.

Following drying, the solid product is recovered. In the case of chemical feedstocks, the purified product is stored or employed. In the case of final products, the purified material is typically packaged or formulated into a finished pharmaceutical composition.

The Products

The products of this purification method are characterized by a substantial increase in purity, that is a decrease in impurity levels by a factor of at least 2 and preferably at least about 3 as compared to the starting material.

The invention is illustrated by, but in no way limited by, the following examples.

### EXAMPLE 1

Lowering impurity levels in 4-formyl-1,3-benzenedisulfonic acid disodium salt (2,4-DSAD-Na, Compound A) using water/IPA.

75.0 g of 2,4-DSAD-Na was stirred in 180 mL 2-propanol at ambient temperature. 60 mL of water was added and the suspension stirred for 10 minutes. The suspensions was filtered and dried 24 hours at 85° C., under vacuum, to give purified 2,4-DSAD-Na in 88% yield.

The chloride level, as expressed by % weight, was measured by ISE (Ion Selective Electrode). HPLC values are expressed as % area.

| Factor | Before Purification | After Purification |
|---|---|---|
| % 2,4-DSAD-Na | 99.4 | 99.8 |
| (HPLC % area) | | |
| % ArCH(OMe)₂ | 0.0 | 0.0 |
| (HPLC % area) | | |
| % ArCH₂OH | 0.4 | 0.2 |
| (HPLC % area) | | |
| % ArCO₂H | 0.1 | 0.0 |
| (HPLC % area) | | |
| % Chloride | 0.7 | 0.1 |
| ISE (w/w) | | |

### EXAMPLE 2

Lowering impurity levels in 4-formyl-1,3-benzenedisulfonic acid disodium salt (2,4-DSAD-Na) using water.

7.875 kg of 2,4-DSAD-Na was stirred in 8.0 L if water for 15 minutes. The slurry was filtered, then dried under vacuum oven for 24 hours at 70° C. for 24 hours to give purified material in 80% yield.

HPLC (%area) before purification was 99.2% 2,4-DSAD-Na.

HPLC (%area) after purification was 99.7%2,4-DSAD-Na.

### EXAMPLE 3

Lowering the impurity levels in Compound B

1.0 kg of Compound B was stirred on 1.5 L of 2-propanol. 0.5 L of water was added and the suspension stirred for 18 hours. The suspension was filtered, dried at 50° C., under vacuum, to give purified Compound B the yield was 58%.

| Factor | Before Purification | After Purification |
|---|---|---|
| % Compound B | 96.1 | 99.6 |
| (HPLC % area) | | |
| % 2,4-DSAD-Na | 0.4 | 0.2 |
| (HPLC % area) | | |
| % ArCH(OMe)₂ | 2.2 | 0.0 |
| (HPLC % area) | | |
| % ArCH₂OH | 0.7 | 0.1 |
| (HPLC % area) | | |
| % ArCO₂H | 0.2 | 0.2 |
| (HPLC % area) | | |
| % Chloride | 2.5 | 0.1 |
| ISE (w/w) | | |

### EXAMPLE 4

Lowering the impurity levels in Compound B

1.0 kg of Compound B was stirred in 1.5 L of acetone. 0.5 L of water was added and the suspension stirred for 18 hours. The suspension was filtered, dried at 50° C., under vacuum, to give purified Compound B the yield was 54%

| Factor | Before Purification | After Purification |
|---|---|---|
| % Compound B | 96.1 | 99.6 |
| % 2,4-DSAD-Na | 0.4 | 0.2 |
| % ArCH(OMe)₂ | 2.2 | 0.0 |
| % ArCH₂OH | 0.7 | 0.1 |
| % ArCO₂H | 0.2 | 0.2 |
| % Chloride | 2.5 | 0.1 |

### EXAMPLE 5

Lowering impurity levels in 4-formyl-1, 3-benzenedisulfonic acid disodium salt (2,4-DSAD-Na) using water.

10.0 kg of 2,4-DSAD-Na is stirred in 10.0 L water for 75 minutes in a 50 L flask. A sample of solid is taken and analyzed by HPLC and purity is verified to be greater than 99.5%. The slurry is pumped from the flask to a filter. All free liquid is removed and the solid is transferred to a Gruenberg oven, then dried for about 24 hours at 80° C. to give purified material.

1. A method for purifying an aryl sulfonate of formula I 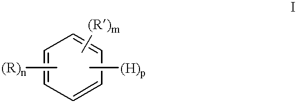
wherein n is an integer from 1 through 3 inclusive, m is an integer from 1 through 3 inclusive and p is an integer 0 through 4 inclusive, with the sum of n+m+p equal to 6, and wherein each R independently represents SO₃H or a salt thereof, and each R′ is an organic substituting group;
which method comprises:
obtaining the aryl sulfonate of formula I as an impure particulate solid containing up to 15% molar impurities;
forming a slurry of said impure particulate solid in a liquid phase comprising water, the composition and quantity of liquid phase selected to dissolve not more than ⅔ of the impure particulate solid;
agitating said slurry,
phase separating said slurry into purified solid phase comprising purified aryl sulfonate and a loaded liquid phase comprising dissolved aryl sufonate and dissolved impurities; and
recovering said purified solid phase.

2. The method according to claim 1 wherein the liquid phase comprises water in admixture with a carrier liquid.

3. The method according to claim 1 wherein the liquid phase consists essentially of water.

4. The method according to claim 2 where in the quantity of liquid phase is selected to dissolve not more than 1/2 of the impure particulate solid.

5. The method according to claim 3 wherein the quantity of liquid phase is selected to dissolve not more than ½ of the impure particulate solid.

6. The method according to claim 1 wherein n is 1.

7. The method according to claim 1 wherein n is 2.

8. The method according to claim 1 wherein m is 1 and R is an aldehyde substituent.

9. The method according to claim 8 wherein the two Rs are both SO₃H.

10. The method according to claim 8 wherein the two Rs are both salts of SO₃H.

11. The method according to claim 8 wherein the aryl sulfonate of formula I is 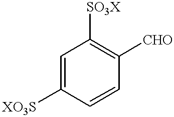
wherein X is hydrogen or a pharmaceutically acceptable cation.

12. The method according to claim 11 wherein X is Na.

13. The method according to claim 7 wherein m is 1 and R¹ is a nitrone substituent.

14. The method according to claim 13 wherein the two Rs are both SO₃H.

15. The method according to claim 13 wherein the two Rs are both salts of SO₃H.

16. The method according to claim 13 wherein the aryl sulfonate of formula I is 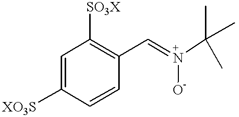
wherein X is hydrogen or a pharmaceutically acceptable cation.

17. The method according to claim 16 wherein X is Na.

18. The method according to claim 1 wherein said recovering comprises drying.

19. A method for purifying an aryl sulfonate of formula II 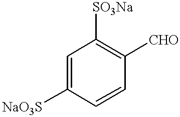
which comprises:
obtaining the aryl sulfonate of formula II as an impure particulate solid,
forming a slurry of said impure particulate solid in from about 0.75 to about 1.25 mL of water per gram of solid,
agitating said slurry for from 15 to 120 minutes,
phase separating the slurry into a purified solid phase and a liquid phase and drying the purified solid phase.

20. A purified aryl sulfonate prepared by the method according to claim 1.

21. A purified aryl sulfonate prepared by the method according to claim 12.

22. A purified aryl sulfonate prepared by the method according to claim 17.

23. A purified aryl sulfonate prepared by the method according to claim 19.
